(19) United States
(12) Patent Application Publication
(10) Pub. No.: US 2023/0071105 A1
(43) Pub. Date: Mar. 9, 2023

(54) JACKFRUIT FLOUR FOR REDUCING CHEMOTOXICITY AND USES THEREOF

(71) Applicant: GOD'S OWN FOOD SOLUTIONS PVT. LTD, Ernakulam Kerala (IN)

(72) Inventors: James JOSEPH, Aluva, Kerala (IN); Thomas VARUGHESE, Netoor, Kerala (IN)

(21) Appl. No.: 17/795,860

(22) PCT Filed: Feb. 1, 2021
(86) PCT No.: PCT/IN2021/050104

§ 371 Date: Jul. 27, 2022

(30) Foreign Application Priority Data
Jan. 31, 2020 (IN) 202041004456

(51) Int. Cl. A61K 36/60; A23L 19/00; A23L 2/52; A61P 37/04; A61K 38/19; A61K 9/00; A61P 7/00; A61K 31/337; A61K 47/60

(52) U.S. Cl. CPC A61K 36/60; A23L 19/01; A23L 2/52; A61P 37/04; A61K 38/193; A61K 9/0095; A61P 7/00; A61K 31/337; A61K 47/60; A23V 2002/00

(57) ABSTRACT

The present disclosure discloses a method for preventing or treating chemotherapy induced leukopenia in a subject said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject wherein the jackfruit flour is introduced within 22-25 hours from the start of administering the chemotherapeutic agent and wherein the jackfruit flour comprises a combination of flour of jackfruit fruits, seeds and strands. The present disclosure also discloses jackfruit flour and jackfruit flour in the form of fortified flour and fortified beverage for use in preventing or treating chemotherapy induced leukopenia.

### FIELD OF INVENTION

The present disclosure relates to the field of cancer therapy. Particularly, the present disclosure discloses the use of jackfruit flour in reducing chemotoxicity during cancer therapy.

### BACKGROUND OF INVENTION

Chemotherapy, a standard therapy for cancer, is associated with numerous severe side effects, including acute and chronic toxicity. The intensity of the toxic side effects can be mild (grade 1), moderate (grade 2), severe (grade 3), or life-threatening or disabling (grade 4), according to the WHO classification. Immediate physiological effects can be observed in the patient on their skin and hair, severe internal side effects are observed in the bone marrow, blood, the gastrointestinal tract, the kidneys and subsequently in the heart, lungs and brain. In addition, the chronic effects of chemotherapy include drug resistance, carcinogenicity and infertility. (V, S., 2021. From chemotherapy to biological therapy: A review of novel concepts to reduce the side effects of systemic cancer treatment (Review). [online] PubMed. Available at: <https://pubmed.ncbi.nlm.nih.gov/30570109/>[Accessed 29 Jan. 2021])

Leukopenia is the most common side effect caused by chemotherapy, when white blood cells count drops below 4000. This often leads to limited tolerance to the doses of chemotherapy. It also causes delay in scheduling subsequent chemo cycles and associated with increased morbidity, mortality, and increased treatment costs. Leukopenia may result in febrile neutropenia (FN), which needs hospitalization for evaluation and administration of proper broad-spectrum antibiotics. Leukopenia is also associated with exacerbations of other adverse effects of chemotherapy, like diarrhoea and mucosal ulcers, and respiratory complications like pneumonia, fatigue, electrolytic imbalance, etc. The predictors of chemotherapy induced leukopenia complications includes advanced age, poor performance status, co-morbidities, and low baseline blood cell counts and high chemotherapy dose intensity. (Crawford et al. Chemotherapy induced neutropenia: risks, consequences, new directions for its management. Cancer. 2004, 100, 228-37, doi.org/10.1002/cncr.11882 and Lyman et al. Risk Models for Predicting Chemotherapy-Induced Neutropenia. The Oncologist. 2005, 10, 427-437, doi: 10.1634/theoncologist.10-6-427).

The costs incurred in managing chemotherapy induced leukopenia include both direct medical costs and indirect costs following hospital admissions or intensive care that are borne by the patient. Numerous developments have occurred in the management of leukopenia, which include availability of hematopoietic growth factors and improvement in antibiotic therapy, yet this complication remains a central concern in the delivery of cancer chemotherapy (Crawford et al. Chemotherapy induced neutropenia: risks, consequences, new directions for its management. Cancer. 2004, 100, 228-37, doi.org/10.1002/cncr.11882). Patients with febrile neutropenia may also develop sepsis, pneumonia or foci of infection, such as cellulitis. Approximately 10% of patients admitted with FN develop serious complications leading to death (Kuderer et al.; Mortality, morbidity, and cost associated with febrile neutropenia in adult cancer patients. Cancer. 2006, 106, 2258-2266, doi: 10.1002/cncr.21847). This can be mitigated by reducing chemotherapeutical doses or extending dosing intervals. However, these measures reduce the relative dose intensity (RDI) of chemotherapy, which has the disadvantage of reduced survival rates.

For instance, the patent Application ES2761406T3 describes a compound of the formula N-[2-[(5-bromo-2-doro-pyrimidma-4M) ammo] etMo] carbamate tere-butyl or its pharmaceutically acceptable salt, for use in a method of reducing the effect of chemotherapy on healthy cells in a subject being treated for cancer. The cancer treatment in this case is independent of kinase 4/replication 6 dependent on cyclin (CDK4/6) or abnormal cell proliferation, wherein said healthy cells are hematopoietic stem cells, hematopoietic progenitor cells or renal epithelial cells, the method comprising administering to the subject an effective amount of the compound.

The patent Application US20200222355A1 discloses methods for treating, controlling or mitigating leukopenia and/or thrombocytopenia, comprising administration of compositions comprising monoacetyl-diacyl-glycerol compound for patients undergoing chemotherapy for multiple myeloma, chronic myelogenous leukaemia (CML), acute myeloid leukaemia, or myelodysplastic syndrome.

Similarly, the patent Application US20090203601A1 relates to a method for treating or preventing neutropenia in a patient receiving chemotherapy. The method discloses administration of a multi-polyethylene glycolylated granulocyte colony stimulating factor (multi-PEGylated G-CSF) variant in an amount effective to reduce chemotherapy-induced neutropenia, wherein the multi-PEGylated G-CSF variant is administered to the patient on the same day as chemotherapy.

However, despite the ongoing research in order to develop various methods to reduce chemotoxicity and the side effects of chemotherapy in cancer patients, there still persists a dearth in providing a better and effective solution to the problems associated with the already existing methods with adjuvant, neo adjuvant or palliative chemotherapy and surgical removal of solid tumours, which may show safer and more effective forms of treatment at a cut above to the present conventional methods.

### SUMMARY OF THE INVENTION

In an aspect of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands.

In another aspect of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour.

In another aspect of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 μg; and (c) introducing jackfruit flour as a dietary intervention for 15-20 days for treating chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour.

In another aspect of the present disclosure, there is provided jackfruit flour for use in preventing or treating chemotherapy induced leukopenia, wherein the jackfruit flour comprises a combination of flour of jackfruit fruits, seeds, and strands, and said jackfruit flour comprises: (a) total fats having weight percentage in the range of about 0.3-1.5% in said flour, wherein total fats do not comprise detectable levels of saturated fats; (b) proteins having weight percentage in the range of about 7-9% in said flour; and (c) total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour is administered within 22-25 hours from the start of administering the chemotherapeutic agent for 15-20 days.

In another aspect of the present disclosure, there is provided a fortified flour for use in preventing or treating chemotherapy induced leukopenia, wherein the fortified flour is administered for 15-20 days within 22-25 hours from the start of administering the chemotherapeutic agent, and wherein the fortified flour comprises: (a) at least one flour; and (b) jackfruit flour, wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein jackfruit flour weight percentage in said fortified flour is in the range of about 10-99%.

In another aspect of the present disclosure, there is provided a fortified beverage for use in preventing or treating chemotherapy induced leukopenia, wherein the fortified beverage is administered for 15-20 days within 22-25 hours from the start of administering the chemotherapeutic agent, and wherein the fortified beverage comprises: (a) at least one beverage; and (b) jackfruit flour, wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein jackfruit flour weight percentage in said fortified beverage is in the range of about 10-99%.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the leukopenia prevention percentage and relates to the effect of jackfruit flour included diet (JFD) in the prevention of leukopenia, in accordance with an embodiment of the present disclosure.

FIG. 2 depicts the average monthly sales quantity in vials for filgrastim from Hospital Pharmacy, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

For the purposes of the present document, the term “Jackfruit” is a fruit from the Artocarpus heterophyllus tree, also referred to as “Jackfruit tree” and is native to Western Ghats of India and common in Asia, Africa, and some regions in South America, and it is a member of the Moraceae family.

In the present disclosure, the term “strands” or commonly referred to as “jackfruit rags” or “undeveloped perianth” relates to the latex-like filaments enveloping the white pulp or the flesh of a jackfruit.

In the present disclosure, the term “chemotherapy” is used for directly or indirectly inhibiting the uncontrolled growth and proliferation of cancer cells and the drugs used for achieving the chemotherapy are referred to as “chemotherapeutic agents”. The term “cycle of chemotherapy” refers to the treatment regimen used for directly or indirectly inhibiting the uncontrolled growth and proliferation of cancer cells. The term “treatment” refers to the reduction of the severity of the condition using medication and nutritional supplements. In the present disclosure, the terms “treatment” and “reducing” are interchangeably used. The term “leukopenia” refers to a condition in a subject wherein the white blood cells (also known as leukocytes) in the blood decreases below the count of 4000. The term “febrile neutropenia” refers to a condition in a subject wherein there is a dramatic decrease in a particular type of white blood cells in the blood called granulated neutrophils and this results in high fever and may lead to eventual death.

The term “pegfilgrastim” and “pegylated filgrastim” refers to the covalent attachment or amalgamation of polyethylene glycol with the hemopoietic growth factor stimulating drug called filgrastim.

Further in the present disclosure, the term “fortified flour” refers to flour (such as, wheat for instance) to which various other nutrients or other flours are added to improve its nutritive value. Similarly, the term “fortified beverage” refers to liquid intended for human consumption to which various other nutrients or flours are added to improve its nutritive value.

The term “fats” refers to esters of three fatty acid chains and glycerol and the term “saturated fats” refers to type of fats in which the fatty acids have single bonds.

The term “dietary fiber” refers to the indigestible portion of food derived primarily from plant sources. It mainly comprises “soluble fiber” and “insoluble fiber”. Soluble fiber dissolves in water and is fermented in the colon. It also provides binding characteristics. Insoluble fiber does not dissolve in water, is metabolically inert and adds bulk to food.

The term “subject” refers to any animal (e.g., a mammal), including, but not limited to, humans, non-human primates, rodents, and the like, which is to be the recipient of a particular treatment as described herein.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a time period range of about 22-25 hours should be interpreted to include not only the explicitly recited limits of about 22 hours to about 25 hours, but also to include sub-ranges, such as 22-24 hours, 23-24 hours, and so forth, as well as individual time intervals, within the specified ranges, such as 23.5 hours, and 24.5 hours, for example.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

As discussed in the background section of the present disclosure, in addition to the destruction of tumor cells, cytotoxic chemotherapy suppresses the hematopoietic system and results in impaired host protective mechanisms. The most common and severe side effect of chemotherapeutic agents is leukopenia, which refers to the decrease in leukocytes or white blood cells. Severe leukopenia may result in a dramatic reduction of neutrophil granulocytes also called febrile neutropenia, which needs hospitalization for evaluation and administration of proper broad-spectrum antibiotics. Treatment options of leukopenia and neutropenia include the prophylactic use of colony-stimulating factors like pegfilgrastim. However, repeated admissions of such colony stimulating factors to manage leukopenia also escalate the costs incurred and reduce the quality of life in patients. Current treatment for leukopenia involves the subcutaneous administration of pegfilgrastim after 24 hours of chemotherapy. Studies conducted indicate that only 43.6% of such treatment regimen result in effective treatment of febrile neutropenia. This data suggests that there is an unmet need to prevent and treat chemotherapy induced leukopenia. On the increase in severity of leukopenia even after pegfilgrastim, hemopoietic growth factors like filgrastim, are given once leukopenia sets in to prevent subsequent febrile neutropenia and eventual death.

In order to overcome the limitations associated with the existing methods and drugs available for treating chemotherapy induced leukopenia, the present disclosure provides a method that uses plant-based product to prevent or treat chemotherapy induced leukopenia. In particular, the present disclosure provides a method that uses jackfruit flour as a dietary intervention. Jackfruit comprises an abundance of nutrients including carbohydrates, proteins, vitamins, minerals, and phytochemicals. Several parts of jackfruit tree including fruits, leaves and bark have been extensively used in traditional medicine due to its anticarcinogenic, antimicrobial, antifungal, anti-inflammatory, wound healing, and hypoglycemic effects. The present disclosure discloses that the inclusion of jackfruit flour in the regular diet of those receiving chemotherapy, could prevent chemotoxicity and leukopenia. The present disclosure also indicates that patients who develop leukopenia and febrile neutropenia post-treatment with chemotherapeutic agents and pegfilgrastim can be treated by introducing jackfruit flour as a dietary intervention. In a scenario where the white blood cell count in the subject is below 4000, then filgrastim may also be added in addition to chemotherapeutic agents, pegfilgrastim, and jackfruit flour. Overall, the present disclosure thus discloses a method for preventing and reducing chemotoxicity by introducing jackfruit flour as a dietary intervention in subjects. The mechanism of leukopenia prevention effect of jackfruit can be attributed to the properties of pectin and amino acids present in the jackfruit. Pectin comprises monosaccharides such as galacturonic acid, glucose, arabinose, galactose, and rhamnose and exhibits antioxidant properties that reduce leukopenia and the protection of intestinal crypt stem cells during chemotherapy.

In the present disclosure, the step of introducing jackfruit flour in the disclosed range as a dietary intervention in the subject undergoing chemotherapy, is very crucial in preventing and treating chemotherapy induced leukopenia without causing any adverse reactions in the subject. Also, the method of the present disclosure that deploys the jackfruit flour as a dietary intervention to prevent or treat chemotherapy induced leukopenia helps in reducing the dose of the drugs in the subject, thus overcoming all the limitations associated with the use of the drugs alone. The jackfruit flour is introduced in the subject in the form of a fortified flour or a fortified beverage. Overall, the present disclosure demonstrates the combined effect of jackfruit flour in combination with pegfilgrastim and optionally filgrastim, resulting in complete avoidance of leukopenia, as compared to the individual effect of pegfilgrastim and chemotherapeutic agents, respectively.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands. In another embodiment of the present disclosure, administering pegfilgrastim of 6 mg to the subject within a time period in the range of 23-24 hours after the end of administering the chemotherapeutic agent. In yet another embodiment of the present disclosure, administering pegfilgrastim of 6 mg to the subject within a time period of 24 hours after the end of administering the chemotherapeutic agent.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 16-19 days for preventing chemotherapy induced leukopenia in the subject, wherein the jackfruit flour is introduced within 23-24 hours from the start of administering the chemotherapeutic agent, and wherein the jackfruit flour comprises a combination of flour of jackfruit fruits, seeds, and strands. In another embodiment of the present disclosure, introducing jackfruit flour as a dietary intervention for 16-18 days for preventing chemotherapy induced leukopenia in the subject, wherein the jackfruit flour is introduced within 24 hours from the start of administering the chemotherapeutic agent, and wherein the jackfruit flour comprises a combination of flour of jackfruit fruits, seeds, and strands.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel. In another embodiment of the present disclosure, the chemotherapeutic agent is albumin bound paclitaxel. In yet another embodiment of the present disclosure, the chemotherapeutic agent is capecitabine.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, wherein the chemotherapeutic agent is administered with at least one excipient selected from the group consisting of a neo adjuvant, an adjuvant, and a palliative.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel, and wherein the chemotherapeutic agent is administered with at least one excipient selected from the group consisting of a neo adjuvant, an adjuvant, and a palliative.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, wherein the jackfruit flour is introduced in the form of a fortified flour.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, wherein the jackfruit flour is introduced in the form of a fortified flour, wherein the fortified flour comprises: (i) at least one flour; and (ii) jackfruit flour, wherein the jackfruit flour weight percentage in said fortified flour is in the range of about 10-99%. In another embodiment of the present disclosure, the jackfruit flour weight percentage in said fortified flour is in the range of about 15-90%. In yet another embodiment of present disclosure, the jackfruit flour weight percentage in said fortified flour is in the range of about 20-85%. In one another embodiment of the present disclosure, the jackfruit flour weight percentage in said fortified flour is in the range of about 25-65%. In an alternate embodiment of the present disclosure, the jackfruit flour weight percentage in said fortified flour is in the range of about 30-50%.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, wherein the jackfruit flour is introduced in the form of a fortified flour, wherein the fortified flour comprises: (i) at least one flour is obtained from the group comprising wheat, barley, rye, rice, millet, oat, acorn, amaranth, banana, buckwheat, cassava, chestnut, chickpea, coconut, maize, corn-starch, flax, plea, potato, sorghum, tapioca, teff, and combinations thereof; and (ii) jackfruit flour, wherein the jackfruit flour weight percentage in said fortified flour is in the range of about 10-99%.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, wherein the jackfruit flour is introduced in the form of a fortified flour, wherein the fortified flour comprises: (i) at least one flour is obtained from the group comprising wheat, barley, rye, rice, millet, oat, acorn, amaranth, banana, buckwheat, cassava, chestnut, chickpea, coconut, maize, corn-starch, flax, plea, potato, sorghum, tapioca, teff, and combinations thereof; and (ii) jackfruit flour, wherein the jackfruit flour weight percentage in said fortified flour is in the range of about 10-99%, and wherein the fortified flour further comprising at least one fortifier selected from the group consisting of vitamins, minerals, hole vegetable extracts or extracts of parts thereof, gums and combinations thereof.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, and wherein the jackfruit flour is introduced in the range of 25-35 grams per day for 15-20 days, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel, and wherein the chemotherapeutic agent is administered with at least one excipient selected from the group consisting of a neo adjuvant, an adjuvant, and a palliative, and the jackfruit flour is introduced in the form of a fortified flour, and wherein the fortified flour comprises: (i) at least one flour; and (ii) jackfruit flour, wherein the jackfruit flour weight percentage in said fortified flour is in the range of about 10-99%.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, wherein the jackfruit flour is introduced in the form of a fortified beverage.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, wherein the jackfruit flour is introduced in the form of a fortified beverage, and wherein the fortified beverage comprises: (i) at least one beverage; and (ii) jackfruit flour, wherein the jackfruit flour weight percentage in said fortified beverage is in the range of about 10-99%. In another embodiment of the present disclosure, the jackfruit flour weight percentage in said fortified beverage is in the range of about 15-95%. In yet another embodiment of the present disclosure, the jackfruit flour weight percentage in said fortified beverage is in the range of about 20-90%. In one another embodiment of the present disclosure, the jackfruit flour weight percentage in said fortified beverage is in the range of about 25-80%. In an alternate embodiment of the present disclosure, the jackfruit flour weight percentage in said fortified beverage is in the range of about 29-60%.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, wherein the jackfruit flour is introduced in the form of a fortified beverage, and wherein the fortified beverage comprises: (i) at least one beverage is selected from the group consisting of water, juices, tea, coffee, and soups; and (ii) jackfruit flour, wherein the jackfruit flour weight percentage in said fortified beverage is in the range of about 10-99%.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, and wherein the jackfruit flour is introduced in the range of 25-35 grams per day for 15-20 days. In another embodiment of the present disclosure, the jackfruit flour is introduced in the range of 26-33 grams per day for 16-19 days. In yet another embodiment of the present disclosure, the jackfruit flour is introduced in the range of 28-31 grams per day for 16-18 days. In one another embodiment of the present disclosure, the jackfruit flour is introduced in the range of 29-30 grams per day for 16-17 days.

In an embodiment of the present disclosure, there is provided a method for preventing or treating chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel, and wherein the chemotherapeutic agent is administered with at least one excipient selected from the group consisting of a neo adjuvant, an adjuvant, and a palliative, and wherein the jackfruit flour is introduced in the range of 25-35 grams per day for 15-20 days, and wherein the jackfruit flour is introduced in the form of a fortified beverage, and wherein the fortified beverage comprises: (i) at least one beverage is selected from the group consisting of water, juices, tea, coffee, and soups; and (ii) jackfruit flour, wherein the jackfruit flour weight percentage in said fortified beverage is in the range of about 10-99%.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.4-1.2% in said flour, wherein total fats do not comprise detectable levels of saturated fats; ii. proteins having weight percentage in the range of about 7.5-8.5% in said flour; and iii. total carbohydrates having weight percentage in the range of about 79-81% in said flour, wherein said carbohydrates comprise dietary fiber having weight percentage in the range of about 13-15% of said flour, and sugars having weight percentage in the range of about 4-8% of said flour, wherein dietary fiber comprises soluble fiber having weight percentage in the range of about 3.5-5.5% of said flour, and insoluble dietary fiber having weight percentage in the range of about 9-10% in said flour. In another embodiment of the present disclosure, the jackfruit flour comprises: i. total fats having weight percentage in the range of about 0.5-1.0% in said flour, wherein total fats do not comprise detectable levels of saturated fats; ii. proteins having weight percentage in the range of about 7.7-8.4% in said flour; and iii. total carbohydrates having weight percentage in the range of about 79.5-81% in said flour, wherein said carbohydrates comprise dietary fiber having weight percentage in the range of about 13.5-14.5% of said flour, and sugars having weight percentage in the range of about 5-7% of said flour, wherein dietary fiber comprises soluble fiber having weight percentage in the range of about 3.7-4.2% of said flour, and insoluble dietary fiber having weight percentage in the range of about 9.2-9.7% in said flour.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5.5-6.5 mg to the subject within a time period in the range of 23-24 hours after the end of administering the chemotherapeutic agent; and (c) introducing jackfruit flour as a dietary intervention for 16-19 days for preventing chemotherapy induced leukopenia in the subject, wherein the jackfruit flour is introduced within 23-24 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour.

In an embodiment of the present disclosure, there is provided a method for preventing chemotherapy induced leukopenia in a subject, said method comprising: (a) administering at least one chemotherapeutic agent to a subject; (b) administering pegfilgrastim of 6 mg to the subject within a time period of 24 hours after the end of administering the chemotherapeutic agent; and (c) introducing jackfruit flour as a dietary intervention for 17-19 days for preventing chemotherapy induced leukopenia in the subject, wherein the jackfruit flour is introduced within 24 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel. In another embodiment of the present disclosure, the chemotherapeutic agent is albumin bound paclitaxel.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein chemotherapeutic agent is administered with at least one excipient selected from the group consisting of a neo adjuvant, an adjuvant, and a palliative.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel, and wherein the chemotherapeutic agent is administered with at least one excipient selected from the group consisting of a neo adjuvant, an adjuvant, and a palliative.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands, and wherein the jackfruit flour is introduced in the form of a fortified flour.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands, and wherein the jackfruit flour is introduced in the form of a fortified beverage.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands, and wherein the jackfruit flour is introduced in the range of 25-35 grams per day for 15-20 days.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands, and wherein the jackfruit flour is introduced in the form of a fortified flour, and wherein the fortified flour comprises: (i) at least one flour; and (ii) jackfruit flour, wherein the jackfruit flour weight percentage in said fortified flour is in the range of about 10-99%. In another embodiment of the present disclosure, the jackfruit flour weight percentage in said fortified flour is in the range of about 15-90%. In yet another embodiment of present disclosure, the jackfruit flour weight percentage in said fortified flour is in the range of about 20-85%. In one another embodiment of the present disclosure, the jackfruit flour weight percentage in said fortified flour is in the range of about 25-65%. In an alternate embodiment of the present disclosure, the jackfruit flour weight percentage in said fortified flour is in the range of about 30-50%.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands, and wherein the jackfruit flour is introduced in the form of a fortified flour, and wherein the fortified flour comprises: (i) at least one flour is obtained from the group comprising wheat, barley, rye, rice, millet, oat, acorn, amaranth, banana, buckwheat, cassava, chestnut, chickpea, coconut, maize, corn-starch, flax, plea, potato, sorghum, tapioca, teff, and combinations thereof; and (ii) jackfruit flour, wherein the jackfruit flour weight percentage in said fortified flour is in the range of about 10-99%.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands, and wherein the jackfruit flour is introduced in the form of a fortified flour, and wherein the fortified flour comprises: (i) at least one flour is obtained from the group comprising wheat, barley, rye, rice, millet, oat, acorn, amaranth, banana, buckwheat, cassava, chestnut, chickpea, coconut, maize, corn-starch, flax, plea, potato, sorghum, tapioca, teff, and combinations thereof; and (ii) jackfruit flour, wherein the jackfruit flour weight percentage in said fortified flour is in the range of about 10-99%, and wherein the fortified flour optionally further comprising at least one fortifier selected from the group consisting of vitamins, minerals, hole vegetable extracts or extracts of parts thereof, gums and combinations thereof.

In an embodiment of the present disclosure, there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands, and wherein the jackfruit flour is introduced in the range of 25-35 grams per day for 15-20 days, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel, and wherein the chemotherapeutic agent is administered with at least one excipient selected from the group consisting of a neo adjuvant, an adjuvant, and a palliative, and the jackfruit flour is introduced in the form of a fortified flour, and wherein the fortified flour comprises: (i) at least one flour; and (ii) jackfruit flour, wherein the jackfruit flour weight percentage in said fortified flour is in the range of about 10-99%.

In an embodiment of the present disclosure there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands, and wherein the jackfruit flour is introduced in the form of a fortified beverage and said beverage comprises: (a) at least one beverage; and (b) jackfruit flour, wherein the jackfruit flour weight percentage in said fortified beverage is in the range of about 10-99%.

In another embodiment of the present disclosure, the jackfruit flour weight percentage in said fortified beverage is in the range of about 15-90%. In yet another embodiment of present disclosure, the jackfruit flour weight percentage in said fortified beverage is in the range of about 20-85%. In one another embodiment of the present disclosure, the jackfruit flour weight percentage in said fortified beverage is in the range of about 25-65%. In an alternate embodiment of the present disclosure, the jackfruit flour weight percentage in said fortified beverage is in the range of about 30-50%.

In an embodiment of the present disclosure there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands, and wherein the jackfruit flour is introduced in the form of a fortified beverage and said beverage comprises: (i) at least one beverage is selected from the group consisting of water, juices, tea, coffee, and soups; and (ii) jackfruit flour, wherein the jackfruit flour weight percentage in said fortified beverage is in the range of about 10-99%.

In an embodiment of the present disclosure there is provided a method for preventing chemotherapy induced leukopenia in a subject, said method comprising: (a) administering at least one chemotherapeutic agent to a subject; (b) administering pegfilgrastim in the range of 5-7 mg to the subject within a time period in the range of 22-25 hours after the end of administering the chemotherapeutic agent; and (c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein the jackfruit flour comprises a combination of flour of jackfruit fruits, seeds, and strands, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel, and wherein the chemotherapeutic agent is administered with at least one excipient selected from the group consisting of a neo adjuvant, an adjuvant, and a palliative, and wherein the jackfruit flour is introduced in the range of 25-35 grams per day for 15-20 days, and wherein the jackfruit flour is introduced in the form of a fortified beverage, and wherein the fortified beverage comprises: (i) at least one beverage is selected from the group consisting of water, juices, tea, coffee, and soups; and (ii) jackfruit flour, wherein the jackfruit flour weight percentage in said fortified beverage is in the range of about 10-99%.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 m; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5.5-6.5 mg, to the subject within a time period in the range of 23-24 hours after the end of administering the chemotherapeutic agent; (c) checking the white blood cell count in the subject; and administering filgrastim if the white blood cell count is below 4000, wherein the filgrastim is in the range of 300 μg-450 μg; and (d) introducing jackfruit flour as a dietary intervention for 16-18 days to the subject, wherein the jackfruit flour is introduced within 23-24 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 μg; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel. In another embodiment of the present disclosure, the chemotherapeutic agent is albumin bound paclitaxel.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 m-400 μg; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein chemotherapeutic agent is administered with at least one excipient selected from the group consisting of a neo adjuvant, an adjuvant, and a palliative.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 m; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel, and wherein the chemotherapeutic agent is administered with at least one excipient selected from the group consisting of a neo adjuvant, an adjuvant, and a palliative.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 m; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the jackfruit flour is introduced in the form of a fortified flour.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 μg; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the jackfruit flour is introduced in the form of a fortified beverage.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 m-400 μg; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the jackfruit flour is introduced in the range of 25-35 grams per day for 15-20 days.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 μg; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the jackfruit flour is introduced in the form of a fortified flour, and wherein the fortified flour comprises: (i) at least one flour; and (ii) jackfruit flour, wherein the jackfruit flour weight percentage in said fortified flour is in the range of about 10-99%.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 m; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the jackfruit flour is introduced in the form of a fortified flour, and wherein the fortified flour comprises: (i) at least one flour is obtained from the group comprising wheat, barley, rye, rice, millet, oat, acorn, amaranth, banana, buckwheat, cassava, chestnut, chickpea, coconut, maize, corn-starch, flax, plea, potato, sorghum, tapioca, teff, and combinations thereof; and (ii) jackfruit flour, wherein the jackfruit flour weight percentage in said fortified flour is in the range of about 10-99%.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 m; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the jackfruit flour is introduced in the form of a fortified flour, and wherein the fortified flour comprises: (i) at least one flour is obtained from the group comprising wheat, barley, rye, rice, millet, oat, acorn, amaranth, banana, buckwheat, cassava, chestnut, chickpea, coconut, maize, corn-starch, flax, plea, potato, sorghum, tapioca, teff, and combinations thereof; and (ii) jackfruit flour, wherein the jackfruit flour weight percentage in said fortified flour is in the range of about 10-99%, and wherein the fortified flour optionally further comprising at least one fortifier selected from the group consisting of vitamins, minerals, hole vegetable extracts or extracts of parts thereof, gums and combinations thereof.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 μg; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the jackfruit flour is introduced in the range of 25-35 grams per day for 15-20 days, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel, and wherein the chemotherapeutic agent is administered with at least one excipient selected from the group consisting of a neo adjuvant, an adjuvant, and a palliative, and the jackfruit flour is introduced in the form of a fortified flour, and wherein the fortified flour comprises: (i) at least one flour; and (ii) jackfruit flour, wherein the jackfruit flour weight percentage in said fortified flour is in the range of about 10-99%.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 μg; and (d) introducing jackfruit flour as a dietary intervention for 15-20 days to the subject, wherein the jackfruit flour is introduced within 22-25 hours from the start of administering the chemotherapeutic agent, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the jackfruit flour is introduced in the form of a fortified beverage and said beverage comprises: (a) at least one beverage; and (b) jackfruit flour, wherein the jackfruit flour weight percentage in said fortified beverage is in the range of about 10-99%.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 μg; and (d) introducing jackfruit flour as a dietary intervention for 15-20 days to the subject, wherein the jackfruit flour is introduced within 22-25 hours from the start of administering the chemotherapeutic agent; and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the jackfruit flour is introduced in the form of a fortified beverage and said beverage comprises: (a) at least one beverage selected from a group consisting of water, juices, tea, coffee and soups; and (b) jackfruit flour, wherein the jackfruit flour weight percentage in said fortified beverage is in the range of about 10-99%.

In an embodiment of the present disclosure, there is provided a method for treating chemotherapy induced leukopenia in a subject, said method comprising: (a) administering at least one chemotherapeutic agent to a subject; (b) administering pegfilgrastim in the range of 5-7 mg, to the subject within a time period in the range of 22-25 hours after the end of administering the chemotherapeutic agent; (c) checking the white blood cell count in the subject; and administering filgrastim if the white blood cell count is below 4000, wherein the filgrastim is in the range of 300 μg-400 μg; and (d) introducing jackfruit flour as a dietary intervention for 15-20 days to the subject, wherein the jackfruit flour is introduced within 22-25 hours from the start of administering the chemotherapeutic agent; and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour comprises a combination of flour of jackfruit fruits, seeds, and strands, and wherein the jackfruit flour is introduced in the range of 25-35 grams per day for 15-20 days, and wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel, and wherein the chemotherapeutic agent is administered with at least one excipient selected from the group consisting of a neo adjuvant, an adjuvant, and a palliative, and the jackfruit flour is introduced in the form of a fortified beverage, and wherein the fortified beverage comprises: (i) at least one beverage; and (ii) jackfruit flour, wherein the jackfruit flour weight percentage in said fortified beverage is in the range of about 10-99%.

In an embodiment of the present disclosure, there is provided a jackfruit flour for use in preventing or treating chemotherapy induced leukopenia, wherein the jackfruit flour comprises a combination of flour of jackfruit fruits, seeds, and strands, and said jackfruit flour comprises: (a) total fats having weight percentage in the range of about 0.3-1.5% in said flour, wherein total fats do not comprise detectable levels of saturated fats; (b) proteins having weight percentage in the range of about 7-9% in said flour; and (c) total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour is administered within 22-25 hours from the start of administering the chemotherapeutic agent for 15-20 days.

In an embodiment of the present disclosure, there is provided a fortified flour for use in preventing or treating chemotherapy induced leukopenia, wherein the fortified flour is administered within 22-25 hours from the start of administering the chemotherapeutic agent for 15-20 days, wherein the fortified flour comprises: (a) at least one flour; and (b) jackfruit flour, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein jackfruit flour weight percentage in said fortified flour is in the range of about 10-99%.

In an embodiment of the present disclosure, there is provided a fortified flour for use in preventing or treating chemotherapy induced leukopenia, wherein the fortified flour is administered within 22-25 hours from the start of administering the chemotherapeutic agent for 15-20 days, wherein the fortified flour comprises: (a) at least one flour; and (b) jackfruit flour, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein jackfruit flour weight percentage in said fortified flour is in the range of about 10-99%, and wherein the flour is obtained from the group comprising wheat, barley, rye, rice, millet, oat, acorn, amaranth, banana, buckwheat, cassava, chestnut, chickpea, coconut, maize, corn-starch, flax, plea, potato, sorghum, tapioca, teff, and combinations thereof.

In an embodiment of the present disclosure, there is provided a fortified flour for use in preventing or treating chemotherapy induced leukopenia, wherein the fortified flour is administered within 22-25 hours from the start of administering the chemotherapeutic agent for 15-20 days, wherein the fortified flour comprises: (a) at least one flour; and (b) jackfruit flour, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein jackfruit flour weight percentage in said fortified flour is in the range of about 10-99%, and wherein the fortified flour optionally further comprises at least one fortifier selected from the group consisting of vitamins, minerals, hole vegetable extracts or extracts of parts thereof, gums and combinations thereof.

In an embodiment of the present disclosure, there is provided a fortified flour for use in preventing or treating chemotherapy induced leukopenia, wherein the fortified flour is administered within 22-25 hours from the start of administering the chemotherapeutic agent for 15-20 days, wherein the fortified flour comprises: (a) at least one flour; and (b) jackfruit flour, and wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein jackfruit flour weight percentage in said fortified flour is in the range of about 10-99%, and wherein the flour is obtained from the group comprising wheat, barley, rye, rice, millet, oat, acorn, amaranth, banana, buckwheat, cassava, chestnut, chickpea, coconut, maize, corn-starch, flax, plea, potato, sorghum, tapioca, teff, and combinations thereof, and wherein the fortified flour optionally further comprises at least one fortifier selected from the group consisting of vitamins, minerals, hole vegetable extracts or extracts of parts thereof, gums and combinations thereof.

In an embodiment of the present disclosure, there is provided a fortified beverage for use in preventing or treating chemotherapy induced leukopenia, wherein the fortified beverage within 22-25 hours from the start of administering the chemotherapeutic agent for 15-20 days, and wherein the fortified beverage comprises: (a) at least one beverage; and (b) jackfruit flour, wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein jackfruit flour weight percentage in said fortified beverage is in the range of about 10-99%.

In an embodiment of the present disclosure, there is provided a fortified beverage for use in preventing or treating chemotherapy induced leukopenia, wherein the fortified beverage is administered within 22-25 hours from the start of administering the chemotherapeutic agent for 15-20 days, and wherein the fortified beverage comprises: (a) at least one beverage; and (b) jackfruit flour, wherein the jackfruit flour comprises: i. total fats having weight percentage in the range of about 0.3-1.5% in said flour, wherein total fats do not comprise detectable levels of saturated fats; ii. proteins having weight percentage in the range of about 7-9% in said flour; and iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein jackfruit flour weight percentage in said fortified beverage is in the range of about 10-99%, and wherein the beverage is selected from the group consisting of water, juices, tea, coffee, and soups.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

The working and non-working examples as depicted in the forthcoming sections highlight the importance of jackfruit flour as a dietary intervention for use in preventing and treating chemotherapy induced leukopenia. The jackfruit flour and the process of obtaining the jackfruit flour are disclosed in the Indian Patent Application 201847013577, which is incorporated in its entirety in the present disclosure.

The examples in the forthcoming sections demonstrate the combined effect of the jackfruit flour in combination with colony stimulating factors such as pegfilgrastim and optionally with hematopoietic growth factors such as filgrastim along with a regular diet of the subject in preventing and treating chemotherapy induced leukopenia of the present disclosure, as compared to their individual effect. It is further specified that in the method of the present disclosure, the step of introducing the jackfruit flour as a dietary intervention is critical so as to successfully prevent the chemotherapy induced leukopenia. The absence of jackfruit flour as described in the examples substantially affects the health of the patients undergoing chemotherapy, and hence fails to exhibit the desired effect.

### Materials and Methods

Method: This disclosure discloses a retrospective cohort study conducted among patients undergoing chemotherapy for solid tumors at the Department of Oncology, Renai Medicity Hospital, Palarivattom, Cochin, Kerala, India.

Statistical analysis: Data was checked for completeness and consistency and entered in IBM SPSS version 20 software. Descriptive statistics like percentages and mean were used. Analysis was done using ANOVA test for comparison between study and control group. P value less than 0.05 was considered statistically significant.

### Example 1

The study group comprised of 50 consecutive subjects, wherein jackfruit flour was introduced in their regular diet. The control group comprised of 50 subjects following their regular diet with no specific dietary interventions. Regular diet of patients in both groups includes vegetarian and non-vegetarian dishes with importance to fish accompanied with rice meal or meals prepared with rice or wheat flour. The two groups of patients matched with regards to gender, age, types of solid tumors, types of chemotherapy drugs used, pegylated filgrastim used and chemotherapy protocols followed in the oncology clinic.

The patients received chemotherapy for solid tumours that included cancers of the breasts, lungs, buccal mucosa, tongue, pharynx, cancers of head and neck, stomach, esophagus, gastro esophageal junction, colon, rectum, pancreas, cancers of gastro intestinal system, bladder, ovaries, uterus, cervix, soft tissue sarcomas. The chemotherapeutic drugs used for the treatment included, but not limited to taxanes, anthracyclines, holoxan, capecitabine, platinums, targeted chemotherapeutic drugs like herceptin, bevacizumab, nimotuzumab. Chemotherapy was given in cycles with the neo adjuvant, adjuvant or palliative indications depending on the health of the patients. Those who received less than 3 cycles were excluded from either study group. The study was approved on Sep. 6, 2019 by the Renai Medicity Hospital institutional ethics committee (Reg. No. ECR/1113/Inst/KL/2018. Ref No. HR/RIMS/158/2019).

Apart from the regular diet, patients in the study arm were advised to include 30 grams of jackfruit flour to rice or wheat flour they used to prepare meals in equal divided doses with breakfast and dinner, without changing any other elements of day to day food habits. Jackfruit flour can also be introduced to the patients in the form of fortified beverages, wherein 10 grams of jackfruit flour would be dissolved in 200 mL of beverage such as water, juices, tea, coffee, or soups. The beverage would be served three times a day totaling to 30 grams of jackfruit flour per day. Jackfruit Flour is a formulation described in Indian Patent Application No. is 201847013577 which is incorporated in its entirety in the present disclosure. Jackfruit flour comprises a combination of flour of dehydrated mature jackfruit fruits, seeds, and strands fortified with protein and soluble fiber from other parts of jackfruit.

Study group patients were given coupons to buy jackfruit flour packs from the local grocery shops and records were maintained. Patients were advised to take 30 grams of jackfruit flour per day along with staple food from first day of chemotherapy through the last day of each cycle of 21 days, till the completion of the course. Blood examination for haemoglobin, white blood cell count and platelet counts were done on 5^{th}, 7^{th}, 9^{th} and 12^{th} day as per standard protocol to watch out for any signs of leukopenia. Patients presented with white blood cell count of less than 4000 got daily doses of filgrastim till their white blood cell count reached above 4000. Data of filgrastim use is maintained in hospital patient records and in the pharmacy. No extra invasive or non-invasive tests or procedures were conducted on the patient for the study.

In the study arm, addition of jackfruit flour to the diet was the patient's choice however the benefits of the dietary intervention of jackfruit flour were explained to all the participants. Adoption for jackfruit flour was high as it was part of the traditional diet with no known adverse effects. Those who refused the dietary intervention initially, in the prospective group, were studied separately. The final pathology report was critically evaluated in all the subjects who received neoadjuvant chemotherapy to assess any adverse or beneficial effects of the dietary intervention of jackfruit flour on the tumor impacting chemo response.

### Results:

After excluding patients who received less than 3 cycles of chemotherapy, analysis was done for a total of 87 patients with 37 patients in study group and 50 patients in control group. Table 1 showed that about 70.3% of the participants were females in study group and 74.0% of the participants were females in control group. About 62.2% and 50% of the participants received neoadjuvant chemotherapy in study group and control group, respectively. The mean age of participants in the study group and the control group were 53.16±11.06 years and 56.96±12.16 years respectively (Table 1).

TABLE 1
Baseline Characteristics of Participants

| Variable | Category | Study | Control |
|---|---|---|---|
| Gender | Male | 11 (29.7%) | 13 (26.0%) |
| | Female | 26 (70.3%) | 37 (74.0%) |
| Therapy given | Adjuvant | 12 (32.4%) | 21 (42.0%) |
| | Neo adjuvant | 23 (62.2%) | 25 (50.0%) |
| | Palliative | 2 (5.4%) | 4 (8.0%) |
| Age | | 53.16 ± 11.06 | 56.96 ± 12.16 |
| (Mean ± SD) | | | |

The participants in control group developed leukopenia in 37 out of 263 cycles of chemotherapy received and 14 out of 232 cycles of chemotherapy in the study group. Six patients in the study group and twenty patients in the control group developed leukopenia, respectively. Twenty patients in the control group required 105 vials of filgrastim, whereas only six patients in the study group required a total of 38 vials of filgrastim (Table 2).

TABLE 2
Descriptive Statistics for Patients in Study and Control Group

| | Study | Control |
|---|---|---|
| | group | group |
| Total number of cycles of chemotherapy given | 232 | 263 |
| Number of cycles in which leukopenia was | 14 | 37 |
| present | | |
| Number of vials of filgrastim taken | 38 | 105 |
| Number of patients for whom leukopenia | 6 | 20 |
| occurred | | |

After excluding those cycles in study group patients, where jackfruit flour was not taken, none of the patients in study group developed leukopenia in any cycle out of the 211 cycles of chemotherapy (Table 3).

TABLE 3
Descriptive Statistics for Patients in Study and Control
Group, after excluding those cycles from study group
patients where jackfruit flour was not taken

| | Study | Control |
|---|---|---|
| | group | group |
| Total number of cycles of chemotherapy given | 211 | 263 |
| Number of cycles in which leukopenia was | 0 | 37 |
| present | | |
| Number of vials of filgrastim taken | 0 | 105 |
| Number of patients for whom leukopenia | 0 | 20 |
| occurred | | |

There was no significant difference in the mean number of cycles of chemotherapy received by patients between the two groups, study group (6.27±2.58) vs control group (5.26±2.52). Significant difference was not found (P=0.15) between the mean number of vials of filgrastim taken per patient in study group (1.02±3.25) and control group (2.10±3.59) respectively (Table 4).

TABLE 4
Difference Between Parameters in Study and Control Group

| | Study group | Control group | |
|---|---|---|---|
| | (mean ± SD) | (mean ± SD) | p-value |
| Number of cycles of | 6.27 ± 2.58 | 5.26 ± 2.52 | 0.07 |
| chemotherapy per patient | | | |
| Number of vials of | 1.02 ± 3.25 | 2.10 ± 3.59 | 0.15 |
| filgrastim taken per patient | | | |

After excluding the cycles in study group in which jackfruit flour was not taken as a dietary intervention, the mean number of cycles of chemotherapy received by patients in the study group was 5.70±2.73 and it was not significantly different (P=0.44) from the patients in the control group (5.26±2.52). Also, mean number of cycles in which leukopenia occurred in the study group (0.00±0.00, P=0.00) and mean number of vials of filgrastim taken per patient in the study group (0.00±0.00, P=0.00) were significantly lesser as compared to the control group (Table 5).

TABLE 5
Difference between parameters in study and control
group, after excluding those cycles from
study group patients where jackfruit flour was not taken)

| | Study group | Control group | |
|---|---|---|---|
| | (mean ± SD) | (mean ± SD) | p-value |
| Number of cycles of | 5.70 ± 2.73 | 5.26 ± 2.52 | 0.44 |
| chemotherapy per patient | | | |
| Number of cycles in which | 0 | 0.74 ± 1.05 | 0.00 |
| leukopenia was present per | | | |
| patient | | | |
| Number of vials of | 0 | 2.10 ± 3.59 | 0.00 |
| filgrastim taken per patient | | | |

In the study group, two patients could not take any oral food due to their cancer and could not take jackfruit flour as a dietary intervention in any cycles. Four patients switched to the jackfruit flour diet only after they experienced leukopenia on a diet without jackfruit flour.

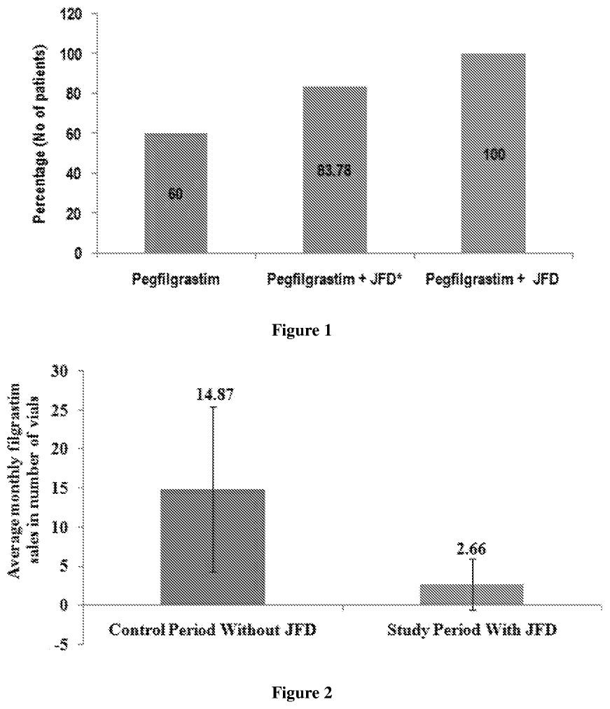

Based on the above results and FIG. 1, it can be observed that the patients in study group who received jackfruit flour as a dietary intervention in all cycles did not experience leukopenia. Therefore, it can be inferred that introducing the jackfruit flour as a dietary intervention to the subject undergoing chemotherapy in combination with the step of administration of prophylactic pegfilgrastim, helped in the complete prevention of chemotherapy induced leukopenia. Hence, the jackfruit flour also helped in enhancing the clinical utility of pegfilgrastim as depicted in FIG. 1.

This was also shown in FIG. 2 where monthly average sales of filgrastim from hospital pharmacy reduced from 14.87 vials to just 2.66 vials after the introduction of jackfruit flour as a dietary intervention. Contrary to this, the patients who didn't take jackfruit flour required a greater number of vials of filgrastim. Around fifty percent of the subjects in either arm received neoadjuvant chemotherapy. It was also observed that the response to chemotherapy was better with more complete pathological responses in the study arm in which the jackfruit flour was introduced as dietary intervention, as compared to the control group in which no jackfruit flour was introduced as a dietary intervention. The results further suggest the absence of adverse response of jackfruit flour to the actions of the drugs on cancer.

There is an improvement in the quality of life and psychological satisfaction when leukopenia and neutropenia are reduced during chemotherapy. The data in FIG. 1 thus exemplifies the added value of jackfruit flour as a dietary intervention due to its ability to improve leukopenia and, subsequently, febrile neutropenia induced by chemotherapy. The leukopenia prevention effect of jackfruit flour is proved in the present disclosure by the fact that the mean number of cycles in which leukopenia developed and the number of vials of filgrastim required was significantly lower in the patients who received jackfruit flour diet, as compared to the patients who did not receive the jackfruit flour as a dietary intervention. It is further substantiated from FIG. 2, that the introduction of the jackfruit flour as a dietary intervention in the subject reduces the treatment cost by avoiding administration of hematopoietic growth factor, antibiotics, and repeated hospital admissions.

### Example 2

A further detailed study was conducted on the subset of 60 patients undergoing albumin bound paclitaxel chemotherapy for solid tumours due to breast cancer at Renai Medicity Hospital, Cochin, India since June 2018. Patients in both the groups were also advised to undergo physical exercise such as brisk walking for a minimum of 5 kms while on chemotherapy and required to take a minimum of 2 litres of water a day. The patients in both the study group and the control group also received 6 mg of pegfilgrastim treatment for prophylaxis after 24 hours of the beginning of the administration of albumin bound paclitaxel (chemotherapeutic agent).

In the study group, 30 consecutive subjects were supplemented with 30 grams of the jackfruit flour diet in their regular diet and their white blood cell count was observed on the on 5^{th}, 7^{th}, 9^{th} and 12^{th} day after chemotherapy. In the control group, 30 patients did not receive jackfruit flour as an additional dietary intervention. During the study, in patients with severe cases, chemotherapeutic agents such as platinums, and anthracyclines were also administered based on their white blood cell count and tests such as immunohistochemistry. Post chemotherapy, capecitabine was also administered to the patients for 14 days. Patients admitted in the hospital after their white blood cell count was less than 4000, indicating chemotherapy induced leukopenia and febrile neutropenia, further received filgrastim treatment.

### Results

Analysis was done for a total of 60 patients with 30 patients in study group and 30 patients in the control group. The results of the analysis done for both study group and control group are provided in Table 6. The results indicated that two out of 30 patients in the study group developed chemotherapy induced leukopenia and received a total of 10 vials of filgrastim and jackfruit flour for leukopenia treatment until their white blood cell count increased above 4000. On the other hand, in the control group, 12 out of 30 patients developed chemotherapy induced leukopenia and they received a total of 72 vials of filgrastim.

TABLE 6
Descriptive Statistics for Patients in Study and Control
Group, after excluding those cycles from study group
patients where jackfruit flour was not taken

| | Study | Control |
|---|---|---|
| | group | group |
| Total number of cycles of chemotherapy given | 140 | 151 |
| Number of cycles in which leukopenia was | 2 | 24 |
| present | | |
| Number of vials of filgrastim taken | 10 | 72 |
| Number of patients for whom leukopenia | 2 | 12 |
| occurred | | |

Referring to Table 6, in the study group the two patients who developed leukopenia, were further treated for leukopenia with the administration of filgrastim and 30 grams of jackfruit flour. After excluding the cycles in the study group where patients did not take jackfruit flour in their diet, the results indicated that no patients in the study group developed leukopenia. This was further exemplified by the comparison of the mean number of cycles in which patients developed chemotherapy induced leukopenia (P=0.00) and the subsequent number of vials of filgrastim taken per cycle (P=0.00) between the control group and the study group. There is a significant difference in the study groups from the control group.

Thus, it can be concluded from the above results that the inclusion of jackfruit flour as a dietary intervention prevents chemotherapy-induced leukopenia effectively in patients undergoing chemotherapy with Albumin Bound Paclitaxel along with pegfilgrastim and can be used to treat patients who develop leukopenia in combination with filgrastim. It can also be inferred that the step of introducing jackfruit flour in the disclosed range as a dietary intervention in the subject undergoing chemotherapy, is very crucial in preventing and treating chemotherapy induced leukopenia without causing any adverse reactions in the subject. Also, using the jackfruit flour as a dietary intervention reduces the dose of the drugs in the subject, thus overcoming all the limitations associated with the use of the drugs alone.

Overall, the present disclosure describes a method for preventing and treating chemotherapy induced leukopenia by using a combination of at least one chemotherapeutic agent, pegfilgrastim and jackfruit flour in the disclosed order of administration. The present disclosure also demonstrates the combined effect of jackfruit flour in combination with pegfilgrastim, resulting in complete avoidance of leukopenia, as compared to the individual effect of pegfilgrastim and chemotherapeutic agents. Thus, the present disclosure provides a cost-effective treatment of a patient undergoing chemotherapy. The critical aspect of the present disclosure lies in administering 30 grams of jackfruit flour as a dietary intervention directly or in the form of fortified flour or fortified beverage and the resulting prevention or treatment of leukopenia in patients undergoing chemotherapy. This aspect was further demonstrated in the patients in the study arm of example 2, where patients who received jackfruit flour as a dietary intervention did not require subsequent treatment using filgrastim, indicating the absence of leukopenia and neutropenia. Further, it was observed that the patients suffering from chemotherapy induced leukopenia can be treated by filgrastim and introducing jackfruit flour as a dietary intervention. This is exemplified by using the subset of patients in the study arm of example 2, wherein two patients with breast cancer and ovarian cancer, who did not receive the appropriate amount of jackfruit flour as a dietary intervention initially were successfully treated for chemotherapy induced leukopenia by introducing the jackfruit flour and administration of filgrastim vials until their white blood cell count increased above 4000.

### Advantages of the Present Disclosure:

The present disclosure discloses that the addition of jackfruit flour as a dietary intervention can be used for prevention and treatment for chemotherapy induced leukopenia. The present disclosure also demonstrates that the combined effect of jackfruit flour in combination with pegfilgrastim, results in complete avoidance of leukopenia. The complete avoidance of leukopenia, greatly enhances the quality of life and provides a cost effective option for patients undergoing chemotherapy. Treatment and prevention using jackfruit flour as a dietary intervention would further result in benefits for the treating medical personnel and oncologists since they need not waste precious hours for treating complications of chemotherapy.

The present disclosure also demonstrates the impact of treating the side effects of chemotherapeutic agents such as capecitabine in combination with standard neoadjuvant and adjuvant chemotherapy regimens in early breast cancer using a combination of jackfruit flour as a dietary intervention with filgrastim. The present disclosure suggests improved outcomes in survival in breast cancer patients. Among those who have received jackfruit fruit flour as a dietary intervention, no patient experienced these complications, denoting the fact that jackfruit flour can even improve survival in those high-risk groups.

I/We claim:

1. A method for preventing or treating chemotherapy induced leukopenia in a subject, said method comprising:
(a) administering at least one chemotherapeutic agent to a subject;
(b) administering pegfilgrastim in the range of 5-7 mg to the subject within a time period in the range of 22-25 hours after the end of administering the chemotherapeutic agent; and
(c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises a combination of flour of jackfruit fruits, seeds, and strands.

2. A method for preventing chemotherapy induced leukopenia in a subject, said method comprising:
(a) administering at least one chemotherapeutic agent to a subject;
(b) administering pegfilgrastim in the range of 5-7 mg to the subject within a time period in the range of 22-25 hours after the end of administering the chemotherapeutic agent; and
(c) introducing jackfruit flour as a dietary intervention for 15-20 days for preventing chemotherapy induced leukopenia in the subject, wherein the jackfruit flour is introduced within 22-25 hours from the start of administering the chemotherapeutic agent, and wherein the jackfruit flour comprises:
i. total fats having weight percentage in the range of about 0.3-1.5% in said flour, wherein total fats do not comprise detectable levels of saturated fats;
ii. proteins having weight percentage in the range of about 7-9% in said flour; and
iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour.

3. A method for treating chemotherapy induced leukopenia in a subject, said method comprising:
(a) administering at least one chemotherapeutic agent to a subject;
(b) administering pegfilgrastim in the range of 5-7 mg, to the subject within a time period in the range of 22-25 hours after the end of administering the chemotherapeutic agent;
(c) checking the white blood cell count in the subject, and administering filgrastim if white blood cell count is below 4000, wherein administering the filgrastim in the range of 300 μg-400 μg; and
(d) introducing jackfruit flour as a dietary intervention for 15-20 days for treating chemotherapy induced leukopenia in the subject, wherein the jackfruit flour is introduced within 22-25 hours from the start of administering the chemotherapeutic agent; and wherein the jackfruit flour comprises:
i. total fats having weight percentage in the range of about 0.3-1.5% in said flour, wherein total fats do not comprise detectable levels of saturated fats;
ii. proteins having weight percentage in the range of about 7-9% in said flour; and
iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour.

4. The method as claimed in any one of the claims 1-3, wherein the chemotherapeutic agent is selected from the group consisting of taxanes, anthracyclines, holoxan, platinums, herceptin, bevacizumab, nimotuzumab, methotrexate, capecitabine, gemcitabine, and albumin bound paclitaxel.

5. The method as claimed in any one of the claims 1-3, wherein the chemotherapeutic agent is albumin bound paclitaxel.

6. The method as claimed in any one of the claims 1-3, wherein the chemotherapeutic agent is administered with at least one excipient selected from the group consisting of a neo adjuvant, an adjuvant, and a palliative.

7. The method as claimed in any one of the claims 1-3 wherein the jackfruit flour is introduced in the form of a fortified flour.

8. The method as claimed in any one of the claims 1-3, wherein the jackfruit flour is introduced in the form of a fortified beverage.

9. The method as claimed in any one of the claims 1-3, wherein the jackfruit flour is introduced in the range of 25-35 grams per day for 15-20 days.

10. The method as claimed in claim 1, wherein said jackfruit flour comprises:
(a) total fats having weight percentage in the range of about 0.3-1.5% in said flour, wherein total fats do not comprise detectable levels of saturated fats;
(b) proteins having weight percentage in the range of about 7-9% in said flour; and
(c) total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour.

11. The method as claimed in claim 7, wherein the fortified flour comprises:
(a) at least one flour; and
(b) jackfruit flour, wherein the jackfruit flour weight percentage in said fortified flour is in the range of about 10-99%.

12. The method as claimed in claim 8, wherein the fortified beverage comprises:
(a) at least one beverage; and
(b) jackfruit flour, wherein the jackfruit flour weight percentage in said fortified beverage is in the range of about 10-99%.

13. The method as claimed in claim 11, wherein the flour is obtained from the group comprising wheat, barley, rye, rice, millet, oat, acorn, amaranth, banana, buckwheat, cassava, chestnut, chickpea, coconut, maize, cornstarch, flax, plea, potato, sorghum, tapioca, teff, and combinations thereof.

14. The method as claimed in claim 11, optionally further comprising at least one fortifier selected from the group consisting of vitamins, minerals, hole vegetable extracts or extracts of parts thereof, gums and combinations thereof.

15. The method as claimed in claim 12, wherein the beverage is selected from the group consisting of water, juices, tea, coffee, and soups.

16. Jackfruit flour for use in preventing or treating chemotherapy induced leukopenia, wherein the jackfruit flour comprises a combination of flour of jackfruit fruits, seeds, and strands, and said jackfruit flour comprises:
(a) total fats having weight percentage in the range of about 0.3-1.5% in said flour, wherein total fats do not comprise detectable levels of saturated fats;
(b) proteins having weight percentage in the range of about 7-9% in said flour; and
(c) total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, wherein the jackfruit flour is administered within 22-25 hours from the start of administering the chemotherapeutic agent for 15-20 days.

17. A fortified flour for use in preventing or treating chemotherapy induced leukopenia, wherein the fortified flour is administered for 15-20 days within 22-25 hours from the start of administering the chemotherapeutic agent, and wherein the fortified flour comprises:
(a) at least one flour; and
(b) jackfruit flour, wherein the jackfruit flour comprises:
i. total fats having weight percentage in the range of about 0.3-1.5% in said flour, wherein total fats do not comprise detectable levels of saturated fats;
ii. proteins having weight percentage in the range of about 7-9% in said flour; and
iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein jackfruit flour weight percentage in said fortified flour is in the range of about 10-99%.

18. The fortified flour as claimed in claim 17, wherein the flour is obtained from the group comprising wheat, barley, rye, rice, millet, oat, acorn, amaranth, banana, buckwheat, cassava, chestnut, chickpea, coconut, maize, cornstarch, flax, plea, potato, sorghum, tapioca, teff, and combinations thereof.

19. The fortified flour as claimed in claim 17, optionally further comprising at least one fortifier selected from the group consisting of vitamins, minerals, hole vegetable extracts or extracts of parts thereof, gums, and combinations thereof.

20. A fortified beverage for use in preventing or treating chemotherapy induced leukopenia, wherein the fortified beverage is administered for 15-20 days within 22-25 hours from the start of administering the chemotherapeutic agent, and wherein the fortified beverage comprises:
(a) at least one beverage; and
(b) jackfruit flour, wherein the jackfruit flour comprises:
i. total fats having weight percentage in the range of about 0.3-1.5% in said flour, wherein total fats do not comprise detectable levels of saturated fats;
ii. proteins having weight percentage in the range of about 7-9% in said flour; and
iii. total carbohydrates having weight percentage in the range of about 78-82% in said flour, wherein said carbohydrates comprise dietary fiber having weight percentage in the range of about 12-16% of said flour, and sugars having weight percentage in the range of about 3-11% of said flour, wherein dietary fiber comprises soluble fiber having weight percentage in the range of about 3-6% of said flour, and insoluble dietary fiber having weight percentage in the range of about 8-12% in said flour, and wherein jackfruit flour weight percentage in said fortified beverage is in the range of about 10-99%.

21. The fortified beverage as claimed in claim 20, wherein the beverage is selected from the group consisting of water, juices, tea, coffee, and soups.

22. The method as claimed in any one of the claims 1-3, wherein the chemotherapeutic agent is capecitabine.

23. A method for preventing or reducing chemotherapy induced leukopenia in a subject, said method comprising:
(a) administering the at least one chemotherapeutic agent to a subject; and
(b) introducing the jackfruit flour as a dietary intervention for 15-20 days for preventing or reducing chemotherapy induced leukopenia in the subject, wherein the jackfruit flour is introduced within 22-25 hours from the start of administering the chemotherapeutic agent.
